(19) United States
(12) Patent Application Publication
(10) Pub. No.: US 2010/0003209 A1
(43) Pub. Date: Jan. 7, 2010

(54) Odor Blocking Water-Absorbent Compositions

(75) Inventors: Volker Braig, Weinheim-Lutzelsachsen (DE); Michael De Marco, Weinheim (DE)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: BASF SE, Ludwigshafen (DE)

(21) Appl. No.: 12/160,851

(22) PCT Filed: Jan. 11, 2007
(86) PCT No.: PCT/EP2007/050235

§ 371 Date: Jul. 14, 2008

(30) Foreign Application Priority Data
Jan. 20, 2006 (EP) 06100673.0

(51) Int. Cl. A61L 2/23

(52) U.S. Cl. 424/76.1

(57) ABSTRACT

The present invention leads to odor-preventing water-absorbing compositions comprising at least one water-absorbing polymer and at least one urease inhibitor, the polymer being a polymer bearing acid groups, the acid groups being from 50 to 65 mol % neutralized and the urease inhibitor content being in the range from 0.0001% to 0.1% by weight, based on the composition, to processes for the production of the composition and also to hygiene articles and their production.

The present invention leads to odor-preventing water-absorbing compositions comprising at least one water-absorbing polymer and at least one urease inhibitor, to processes for their production and also to hygiene articles and their production.

Further embodiments of the present invention are discernible from the claims, the description and the examples. It will be understood that the hereinbefore mentioned and the hereinbelow still to be elucidated features of the present invention's subject matter are utilizable not only in the particular combination indicated but also in other combinations without leaving the realm of the invention.

Water-absorbing polymers are in particular polymers of (co)polymerized hydrophilic monomers, graft (co)polymers of one or more hydrophilic monomers on a suitable grafting base, crosslinked ethers of cellulose or starch, crosslinked carboxymethylcellulose, partially crosslinked polyalkylene oxide or natural products swellable in aqueous fluids, examples being guar derivatives, water-absorbing polymers based on partially neutralized acrylic acid being preferred. Such polymers are used as products capable of absorbing aqueous solutions to manufacture diapers, tampons, sanitary napkins, incontinence products and other hygiene articles, but also as water-retaining agents in market gardening.

Unpleasant odors can arise in hygiene articles during use through decomposition of urea for example. WO-A-98/26808, WO-A-03/053486, JP-A-2001/258934, EP-A-0 739 635 and EP-A-1 034 800 propose various solutions to the problem.

WO-A-98/26808 describes absorbent compositions comprising any desired fluid-absorbing material, an odor-absorbing material and also one or more substances from the group consisting of biocides, urease inhibitors and pH regulators. Crosslinked polyacrylic acids having a degree of neutralization of at least 75 mol % are mentioned as preferred fluid-absorbing materials.

WO-A-03/053486 discloses the use of yucca extract as a urease inhibitor.

JP-A-2001/258934 teaches the use of weakly acidic water-absorbing polymers having a degree of neutralization in the range from 40 to 65 mol % in combination with urease inhibitors. JP-A-2001/258934 contains no pointer to the disproportionate drop in the absorption capacity when using more than 0.1% by weight of urease inhibitor.

EP-A-0 739 635 describes absorbent compositions comprising boric acid salts.

EP-A-1 034 800 describes the use of combinations of an odor-absorbing agent and an oxidizing agent to control unpleasant odors.

The present invention has for its object to provide improved water-absorbing compositions which, having been insulted with urine or other body fluids, reliably prevent unpleasant odors.

The present invention further has for its object to provide odor-preventing water-absorbing compositions which are stable in storage, i.e., which neither discolor nor lose their odor-preventing effect in the course of prolonged storage.

We have found that this object is achieved by compositions comprising at least one water-absorbing polymer and at least one urease inhibitor, the polymer being a polymer bearing acid groups, the acid groups being from 50 to 65 mol % neutralized and the urease inhibitor content being in the range from 0.0001% to 0.1% by weight, based on the composition.

The acid groups of the water-absorbing polymer are preferably from 52 to 63 mol %, more preferably from 54 to 61 mol % and most preferably from 55 to 60 mol % neutralized.

The composition of the present invention comprises typically at least 90% by weight, preferably at least 95% by weight, and more preferably at least 99% by weight of the at least one water-absorbing polymer.

The at least one water-absorbing polymer is preferably a polymer based on a crosslinked acrylic acid.

The at least one water-absorbing polymer is preferably present in the form of surface-postcrosslinked particles.

The water-absorbing polymers are obtained for example by polymerization of a monomer solution comprising
a) at least one ethylenically unsaturated acid-functional monomer,
b) at least one crosslinker,
c) if appropriate one or more ethylenically and/or allylically unsaturated monomers copolymerizable with the monomer a), and
d) if appropriate one or more water-soluble polymers onto which the monomers a), b) and if appropriate c) can be at least partly grafted.

Suitable monomers a) are for example ethylenically unsaturated carboxylic acids, such as acrylic acid, methacrylic acid, maleic acid, fumaric acid and itaconic acid, or derivatives thereof, such as acrylamide, methacrylamide, acrylic esters and methacrylic esters. Acrylic acid and methacrylic acid are particularly preferred. Acrylic acid is most preferable.

The monomers a) and especially acrylic acid comprise preferably up to 0.025% by weight of a hydroquinone half ether. Preferred hydroquinone half ethers are hydroquinone monomethyl ether (MEHQ) and/or tocopherols.

Tocopherol refers to compounds of the following formula:

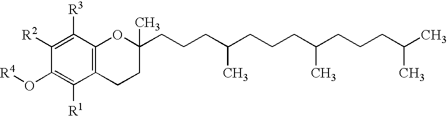

where R¹ is hydrogen or methyl, R² is hydrogen or methyl, R³ is hydrogen or methyl and R⁴ is hydrogen or an acyl radical of 1 to 20 carbon atoms.

Preferred R⁴ radicals are acetyl, ascorbyl, succinyl, nicotinyl and other physiologically tolerable carboxylic acids. The carboxylic acids can be mono-, di- or tricarboxylic acids.

Preference is given to alpha-tocopherol where R¹═R²═R³=methyl, especially racemic alpha-tocopherol. R¹ is more preferably hydrogen or acetyl. RRR-alpha-tocopherol is preferred in particular.

The monomer solution comprises preferably not more than 130 weight ppm, more preferably not more than 70 weight ppm, preferably not less than 10 weight ppm, more preferably not less than 30 weight ppm and especially about 50 weight ppm of hydroquinone half ether, all based on acrylic acid, with acrylic acid salts being counted as acrylic acid. For example, the monomer solution can be produced using an acrylic acid having an appropriate hydroquinone half ether content.

The crosslinkers b) are compounds having at least two polymerizable groups which can be free-radically interpolymerized into the polymer network. Suitable crosslinkers b) are for example ethylene glycol dimethacrylate, diethylene glycol diacrylate, allyl methacrylate, trimethylolpropane triacrylate, triallylamine, tetraallyloxyethane, as described in EP-A-0 530 438, di- and triacrylates, as described in EP-A-0 547 847, EP-A-0 559 476, EP-A-0 632 068, WO-A-93/21237, WO-A-03/104299, WO-A-03/104300, WO-A-03/104301 and DE-A-103 31 450, mixed acrylates which, as well as acrylate groups, comprise further ethylenically unsaturated groups, as described in DE-A-1 03 31 456 and WO-A-04/013064, or crosslinker mixtures as described for example in DE-A-195 43 368, DE-A-196 46 484, WO-A-90/15830 and WO-A-02/32962.

Useful crosslinkers b) include in particular N,N′-methylenebisacrylamide and N,N′-methylenebismethacrylamide, esters of unsaturated mono- or polycarboxylic acids of polyols, such as diacrylate or triacrylate, for example butanediol diacrylate, butanediol dimethacrylate, ethylene glycol diacrylate, ethylene glycol dimethacrylate and also trimethylolpropane triacrylate and allyl compounds, such as allyl(meth)acrylate, triallyl cyanurate, diallyl maleate, polyallyl esters, tetraallyloxyethane, triallylamine, tetraallylethylenediamine, allyl esters of phosphoric acid and also vinylphosphonic acid derivatives as described for example in EP-A-0 343 427. Useful crosslinkers b) further include pentaerythritol diallyl ether, pentaerythritol triallyl ether, pentaerythritol tetraallyl ether, polyethylene glycol diallyl ether, ethylene glycol diallyl ether, glycerol diallyl ether, glycerol triallyl ether, polyallyl ethers based on sorbitol, and also ethoxylated variants thereof. The process of the invention utilizes di(meth)acrylates of polyethylene glycols, the polyethylene glycol used having a molecular weight between 300 and 1000.

However, particularly advantageous crosslinkers b) are di- and triacrylates of 3- to 15-tuply ethoxylated glycerol, of 3- to 15-tuply ethoxylated trimethylolpropane, of 3- to 15-tuply ethoxylated trimethylolethane, especially di- and triacrylates of 2- to 6-tuply ethoxylated glycerol or of 2- to 6-tuply ethoxylated trimethylolpropane, of 3-tuply propoxylated glycerol, of 3-tuply propoxylated trimethylolpropane, and also of 3-tuply mixedly ethoxylated or propoxylated glycerol, of 3-tuply mixedly ethoxylated or propoxylated trimethylolpropane, of 15-tuply ethoxylated glycerol, of 15-tuply ethoxylated trimethylolpropane, of 40-tuply ethoxylated glycerol, of 40-tuply ethoxylated trimethylolethane and also of 40-tuply ethoxylated trimethylolpropane.

Very particularly preferred for use as crosslinkers b) are diacrylated, dimethacrylated, triacrylated or trimethacrylated multiply ethoxylated and/or propoxylated glycerols as described for example in WO-A-03/104301. Di- and/or triacrylates of 3- to 10-tuply ethoxylated glycerol are particularly advantageous. Very particular preference is given to di- or triacrylates of 1- to 5-tuply ethoxylated and/or propoxylated glycerol. The triacrylates of 3- to 5-tuply ethoxylated and/or propoxylated glycerol are most preferred. These are notable for particularly low residual levels (typically below 10 weight ppm) in the water-absorbing polymer and the aqueous extracts of water-absorbing polymers produced therewith have an almost unchanged surface tension (typically not less than 0.068 N/m) compared with water at the same temperature.

The amount of crosslinker b) is preferably at least 0.001 mol %, more preferably at least 0.005 mol % and most preferably at least 0.01 mol % and preferably up to 10 mol %, more preferably up to 5 mol % and most preferably up to 2 mol %, all based on monomer a).

Examples of ethylenically unsaturated monomers c) which are copolymerizable with the monomers a) are acrylamide, methacrylamide, crotonamide, dimethylaminoethyl methacrylate, dimethylaminoethyl acrylate, dimethylaminopropyl acrylate, diethylaminopropyl acrylate, dimethylaminobutyl acrylate, dimethylaminoethyl methacrylate, diethylaminoethyl methacrylate, dimethylaminoneopentyl acrylate and dimethylaminoneopentyl methacrylate.

Useful water-soluble polymers d) include polyvinyl alcohol, polyvinylpyrrolidone, starch, starch derivatives, polyglycols or polyacrylic acids, preferably polyvinyl alcohol and starch.

Polymerization inhibitors, which are preferred, require dissolved oxygen for optimum performance. Therefore, polymerization inhibitors may be freed of dissolved oxygen prior to polymerization by inertization, i.e., flowing an inert gas, preferably nitrogen, through them. The oxygen content of the monomer solution is preferably lowered to less than 1 weight ppm and more preferably to less than 0.5 weight ppm prior to polymerization.

The preparation of a suitable water-absorbing polymer and also further useful hydrophilic ethylenically unsaturated monomers d) are described in DE-A-199 41 423, EP-A-0 686 650, WO-A-01/45758 and WO-A-03/104300.

Water-absorbing polymers are typically obtained by addition polymerization of an aqueous monomer solution with or without subsequent comminution of the hydrogel. Suitable methods of making are described in the literature. Water-absorbing polymers are obtainable for example by
gel polymerization in the batch process or tubular reactor and subsequent comminution in meat grinder, extruder or kneader (EP-A-0 445 619, DE-A-19 846 413)
addition polymerization in kneader with continuous comminution by contrarotatory stirring shafts for example (WO-A-01/38402)
addition polymerization on belt and subsequent comminution in meat grinder, extruder or kneader (DE-A-38 25 366, U.S. Pat. No. 6,241,928)
emulsion polymerization, which produces bead polymers having a relatively narrow gel size distribution (EP-A-0 457 660)
in situ addition polymerization of a woven fabric layer which, usually in a continuous operation, has previously been sprayed with aqueous monomer solution and subsequently been subjected to a photopolymerization (WO-A-02/94328, WO-A-02/94329)
spray polymerization (WO-A-96/40427, DE-A-103 40 253).

The reaction is preferably carried out in a kneader as described for example in WO-A-01/38402, or on a belt reactor as described for example in EP-A-0 955 086.

The acid groups of the hydrogels obtained have been neutralized to an extent in the range from 50 to 65 mol %, preferably to an extent in the range from 52 to 63 mol %, more preferably to an extent in the range from 54 to 61 mol % and even more preferably to an extent in the range from 55 to 60 mol %, for which the customary neutralizing agents can be used, preferably alkali metal hydroxides, alkali metal oxides, alkali metal carbonates or alkali metal bicarbonates and also mixtures thereof. Instead of alkali metal salts it is also possible to use ammonium salts. Sodium and potassium are particularly preferred as alkali metals, but most preference is given to sodium hydroxide, sodium carbonate or sodium bicarbonate and also mixtures thereof. Neutralization is customarily achieved by admixing the neutralizing agent as an aqueous solution or else preferably as a solid material. For example, sodium hydroxide having a water content of distinctly below 50% by weight can be present as a waxy mass having a melting point of above 23° C. In this case, metering as piecegoods or melt at elevated temperature is possible.

Neutralization can be carried out after polymerization, at the hydrogel stage. But it is also possible to neutralize up to 40 mol %, preferably from 10 to 30 mol % and more preferably from 15 to 25 mol % of the acid groups before polymerization by adding a portion of the neutralizing agent to the monomer solution and setting the desired final degree of neutralization only after polymerization, at the hydrogel stage. The monomer solution can be neutralized by admixing the neutralizing agent. The hydrogel may be mechanically comminuted, for example by means of a meat grinder, in which case the neutralizing agent can be sprayed, sprinkled or poured on and then carefully mixed in. To this end, the gel mass obtained can be repeatedly meat-grindered for homogenization. Neutralization of the monomer solution to the final degree of neutralization is preferred.

The neutralized hydrogel is then dried with a belt or drum dryer until the residual moisture content is preferably below 15% by weight and especially below 10% by weight, the water content being determined by EDANA (European Disposables and Nonwovens Association) recommended test method No. 430.2-02 “Moisture content”. Selectively, drying can also be carried out using a fluidized bed dryer or a heated plowshare mixer. To obtain particularly white products, it is advantageous to dry this gel by ensuring rapid removal of the evaporating water. To this end, the dryer temperature must be optimized, the air feed and removal has to be policed, and at all times sufficient venting must be ensured. Drying is naturally all the more simple—and the product all the more white—when the solids content of the gel is as high as possible. The solids content of the gel prior to drying is therefore preferably between 30% and 80% by weight. It is particularly advantageous to vent the dryer with nitrogen or some other nonoxidizing inert gas. Selectively, however, simply just the partial pressure of the oxygen can be lowered during drying to prevent oxidative yellowing processes. But in general adequate venting and removal of the water vapor will likewise still lead to an acceptable product. A very short drying time is generally advantageous with regard to color and product quality.

The dried hydrogel is preferably ground and sieved, useful grinding apparatus typically including roll mills, pin mills or swing mills. The particle size of the sieved, dry hydrogel is preferably below 1000 μm, more preferably below 900 μm and most preferably below 850 μm and preferably above 80 μm, more preferably above 90 μm and most preferably above 100 μm.

Very particular preference is given to a particle size (sieve cut) in the range from 106 to 850 μm. The particle size is determined according to EDANA (European Disposables and Nonwovens Association) recommended test method No. 420.2-02 “Particle size distribution”.

The water-absorbing polymers are then preferably surface postcrosslinked. Useful postcrosslinkers are compounds comprising two or more groups capable of forming covalent bonds with the carboxylate groups of the hydrogel. Suitable compounds are for example alkoxysilyl compounds, polyaziridines, polyamines, polyamidoamines, di- or polyepoxides, as described in EP-A-0 083 022, EP-A-543 303 and EP-A-937 736, di- or polyfunctional alcohols, as described in DE-C-33 14 019, DE-C-35 23 617 and EP-A-450 922, or β-hydroxyalkylamides, as described in DE-A-102 04 938 and U.S. Pat. No. 6,239,230.

Useful surface postcrosslinkers are further said to include by DE-A-40 20 780 cyclic carbonates, by DE-A-198 07 502 2-oxazolidone and its derivatives, such as 2-hydroxyethyl-2-oxazolidone, by DE-A-1 98 07 992 bis- and poly-2-oxazolidinones, by DE-A-198 54 573 2-oxotetrahydro-1,3-oxazine and its derivatives, by DE-A-198 54 574 N-acyl-2-oxazolidones, by DE-A-102 04 937 cyclic ureas, by DE-A-103 34 584 bicyclic amide acetals, by EP-A-1 199 327 oxetanes and cyclic ureas and by WO-A-03/031482 morpholine-2,3-dione and its derivatives.

It is advantageous to use polyvalent cations for surface postcrosslinking as well as surface postcrosslinkers. Useful polyvalent cations include for example divalent cations, such as the cations of zinc, magnesium, calcium and strontium, trivalent cations, such as the cations of aluminum, iron, chromium, rare earths and manganese, tetravalent cations, such as the cations of titanium and zirconium. Possible counterions are chloride, bromide, sulfate, hydrogen sulfate, carbonate, hydrogencarbonate, nitrate, phosphate, hydrogen phosphate, dihydrogen phosphate and carboxylate, such as acetate and lactate. Aluminum sulfate is preferred.

Postcrosslinking is typically carried out by spraying a solution of the surface postcrosslinker onto the hydrogel or onto the dry polymeric powder. The surface postcrosslinker and the polyvalent cation can be sprayed in a common solution or as separate solutions. After spraying, the polymeric powder is thermally dried, and the crosslinking reaction may take place not only before but also during drying.

The spraying with a solution of the crosslinker is preferably carried out in mixers having moving mixing implements, such as screw mixers, paddle mixers, disk mixers, plowshare mixers and shovel mixers. Particular preference is given to vertical mixers and very particular preference to plowshare mixers and shovel mixers. Useful mixers include for example Lödige® mixers, Bepex® mixers, Nauta® mixers, Processall® mixers and Schugi® mixers. Very particular preference is given to employing high-speed mixers, for example of the Schuggi-Flexomix® or Turbolizer® type.

Contact dryers are preferable, shovel dryers more preferable and disk dryers most preferable as apparatus in which thermal drying is carried out. Useful dryers include for example Bepex® dryers and Nara® dryers. Fluidized bed dryers can be used as well.

Drying may take place in the mixer itself, by heating the jacket or introducing a stream of warm air. It is similarly possible to use a downstream dryer, for example a tray dryer, a rotary tube oven or a heatable screw. But it is also possible for example to utilize an azeotropic distillation as a drying process.

Preferred drying temperatures are in the range from 50 to 250° C., preferably in the range from 50 to 200° C. and more preferably in the range from 50 to 150° C. The preferred residence time at this temperature in the reaction mixer or dryer is below 30 minutes and more preferably below 10 minutes.

Suitable urease inhibitors are 2-bromo-2-nitro-1,3-propanediol(bronopol), triclosan, substituted thiophosphoramides, boric acid and its derivatives, hydroxylamine acid derivatives, cystamine and its derivatives, dicumerol and its derivatives, hydroquinone and its derivatives, fluorides, N-alkylureas, N-arylureas, 5-methoxy-2-[[(4-methoxy-3,5-dimethyl-2-pyridinyl)methyl]sulfinyl]-1H-benzimidazole(omeprazole), oxalic acid dihydrazide, polyvinylpyrrolidine-iodine, organic halogen compounds, preferably iodides and bromides, complexing reagents, heavy metal ions, phosphates and polyphosphates.

Suitable urease inhibitors are also described in J. Enz. Inhib. vol. 16 (2001), pages 507 to 516, J. Am. Chem. Soc. vol. 126 (2004), pages 3714 and 3715, J. Biol. Chem. vol.

264 (1989), pages 15835 to 15842, Current Medicinal Chemistry vol. 9 (2002), pages 1323 to 1348, Appl. Biochem. Microbio. vol. 41 (2005), pages 23 to 28, Biochemistry (Moscow) vol. 69 (2004), pages 1344 to 1352, Appl. Biochem. Microbio. vol. 37 (2001), pages 168 to 173, Food Chem. vol. 85 (2004), pages 553 to 558, Chem. Pharm. Bull. vol. 51 (2003), pages 719 to 723, Bioorg. Med. Chem. vol. 12 (2004), pages 1963 to 1968, and J. Enz. Inhib. Med. Chem. vol. 19 (2004), pages 367 to 371.

Preferred urease inhibitors are substituted thiophosphoramides of the formula (I)

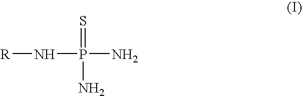

where R is a C₁- to C₃₀-alkyl radical, preferably a C₂- to C₁₀-alkyl radical and more preferably a C₃- to C₅-alkyl radical. The alkyl radicals may be branched or unbranched.

The examples of C₁- to C₁₀-alkyl radicals are methyl, ethyl, n-propyl, isopropyl, n-butyl, isobutyl, sec-butyl, tert-butyl, n-pentyl, isopentyl, n-hexyl, isohexyl, n-heptyl, isoheptyl, n-octyl, isooctyl, n-nonyl, isononyl, n-decyl and isodecyl. The most preferred alkyl radicals are n-propyl and n-butyl.

The substituted thiophosphoramides of the formula (I) are obtainable for example by reacting thiophosphoryl trichloride with alkylamine and ammonia.

The preparation of substituted thiophosphoramides is described in U.S. Pat. No. 5,770,771 for example. Higher purities are achieved when the products are recrystallized in a suitable solvent, an example being toluene.

Preferred urease inhibitors further include substituted phosphoramides of the formula (II)

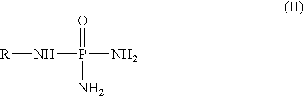

where R is as defined above.

The substituted phosphoramides of the formula (II) are formed for example by hydrolysis of the thiophosphoramides of the formula (I).

Phenyl phosphorodiamidate (CAS No. 7450-69-3) is a further preferred urease inhibitor.

The composition of the present invention comprises typically from 0.0001% to 0.1% by weight, preferably from 0.005% to 0.08% by weight, more preferably from 0.01% to 0.06% by weight and most preferably from 0.015% to 0.045% by weight, of the at least one urease inhibitor.

The compositions of the present invention have an excellent odor-preventing effect and a high absorptive capacity.

The present invention further provides processes for producing the compositions of the present invention, which comprises at least one of the following steps:
i) mixing at least one urease inhibitor with at least one water-absorbing polymer,
ii) grinding at least one urease inhibitor together with at least one water-absorbing polymer,
iii) spraying at least one urease inhibitor onto at least one water-absorbing polymer,
iv) preparing the at least one water-absorbing polymer by solution polymerization of a monomer solution and dissolving or suspending at least one urease inhibitor in the monomer solution.

Mixing may be carried out in any manner and may be effected as early as the production of the water-absorbing polymer, for example in the course of cooling after postcrosslinking or the subsequent sieving, or in a special mixer. Suitable mixers were described above in relation to the postcrosslinking of the water-absorbing polymer.

The manner of grinding is likewise not subject to any restriction. Suitable apparatuses were described above in relation to the comminution of the water-absorbing polymer.

The manner of spraying is not subject to any restriction. The urease inhibitor may be sprayed as a solution or as a melt, for example during the postcrosslinking of the water-absorbing polymer in the mixers mentioned there.

The at least one urease inhibitor is advantageously sprayed as a solution in a suitable solvent. Suitable solvents are water, water-acetone mixtures, water-propylene glycol mixtures and also the solvents and solvent mixtures identified in relation to the postcrosslinking operation. The concentration of the urease inhibitor in the solution is typically in the range from 0.5% to 30% by weight, preferably in the range from 1% to 20% by weight and more preferably in the range from 2% to 10% by weight.

A further embodiment comprises producing a composition according to the present invention that comprises a higher fraction of the at least one urease inhibitor, typically in the range from 1% to 50% by weight, preferably in the range from 5% to 40% by weight and more preferably in the range from 10% to 30% by weight. The highly concentrated composition thus obtained may then be diluted with further water-absorbing polymer to the desired final strength.

The present invention further provides hygiene articles comprising at least one composition according to the present invention, in particular diapers or pads for heavy and/or light incontinence and also sanitary napkins, baby diapers and cat litter, and processes for producing hygiene articles wherein at least one composition according to the present invention is used.

The water-absorbing compositions of the present invention are able to reliably prevent unpleasant odors which can arise in hygiene articles. The compositions of the present invention are stable on storage, so that the odor-controlling effect is still present after prolonged storage, for example 6 months. Furthermore, the compositions of the present invention are free of visible discolorations after prolonged storage.

Methods:

The measurements should be carried out, unless otherwise stated, at an ambient temperature of 23±2° C. and a relative humidity of 50±10%. The water-absorbing polymers are thoroughly commixed prior to the measurement.

Centrifuge Retention Capacity (CRC)

Centrifuge Retention Capacity of the water-absorbing polymeric particles is determined in accordance with the EDANA (European Disposables and Nonwovens Association) recommended test method No. 441.2-02 “Centrifuge retention capacity”.

Odor-Inhibiting Effect

To determine the odor-preventing effect, 2 g of each of the compositions produced above were placed in a 100 ml Erlenmeyer flask and admixed with a freshly prepared solution of 30 mg of urease (from jack beans; lyophilized 5 U/mg for urea assay in serum; Merck KGaA, Germany) and 50 ml of 0.9% sodium chloride solution, the sodium chloride solution containing 8.56 g/l of urea, and sealed with a stopper having an internal diffusion tubelet (Dräger® Röhrchen; ammonia 20/a-D, 20 to 1500 ppm*h). The measurement was discontinued after 6 hours. The test was carried out at 23° C.

### EXAMPLES

### Example 1

Production of Water-Absorbing Polymer (Degree of Neutralization 55 mol %)

3708 g of a 37.3% by weight aqueous sodium acrylate solution were mixed with 867 g of acrylic acid and 1389 g of water and inertized with nitrogen. This mixture was filled into a nitrogen-inertized Werner & Pfleiderer LUK 8.0 K2 kneader (2 sigma shafts) and admixed in succession with 6.2 g of polyethylene glycol diacrylate 400 (diacrylate of a polyethylene glycol having an average molecular weight of 400 g/mol), 15.4 g of a 0.5% by weight aqueous ascorbic acid solution, 12.8 g of a 15% by weight aqueous sodium persulfate solution and 1.5 g of a 2.5% by weight aqueous hydrogen peroxide solution. The kneader was stirred at maximum speed (98 rpm for the faster shaft, about 49 rpm for the slower shaft, ratio about 2:1). Immediately following the addition of hydrogen peroxide, the kneader jacket was heated with hot heat transfer medium at 80° C. 152 g of fine SAP dust having a degree of neutralization of 65 mol % and a particle size of less than 100 μm were added at an internal temperature of 60° C. On reaching the maximum temperature, the jacket heating was switched off and the kneader contents were allowed to react for a further 15 minutes in a supplementary reaction. The gel was cooled down to 65° C. and discharged. The gel was dried at 155° C. for 90 minutes using a loading of 700 g per tray in a circulating air drying cabinet. Following threefold grinding in a roll mill (Gebr. Baumeister LRC 125/70, gap widths 1000 μm, 600 μm, 400 μm), the polymer was sieved to obtain a size cut between 850 and 100 μm.

The Centrifuge Retention Capacity (CRC) of the water-absorbing polymers was 35.7 g/g.

1200 g of this polymer were transferred into a Gebr. Lödige laboratory mixer (M5R model). At room temperature, a mixture of 12 g of 1,2-propanediol, 1.2 g of diethylene glycol diglycidyl ether and 24 g of water was sprayed in via a first nozzle and 12 g of an aluminum sulfate solution (26.8% by weight of Al₂(SO₄)₃ in water) via a second nozzle. The mixer was then rapidly heated to 150° C. and maintained at 150° C. for 30 minutes. After cooling, the polymer was sieved to obtain a size cut between 850 and 100 μm.

The Centrifuge Retention Capacity (CRC) of the surface-postcrosslinked water-absorbing polymers was 28.5 g/g.

### Example 2

Production of Water-Absorbing Polymer (Degree of Neutralization 60 mol %)

3993 g of a 37.3% by weight aqueous sodium acrylate solution were mixed with 761 g of acrylic acid and 1211 g of water and inertized with nitrogen. This mixture was filled into a nitrogen-inertized Werner & Pfleiderer LUK 8.0 K2 kneader (2 sigma shafts) and admixed in succession with 6.3 g of polyethylene glycol diacrylate 400 (diacrylate of a polyethylene glycol having an average molecular weight of 400 g/mol), 15.2 g of a 0.5% by weight aqueous ascorbic acid solution, 12.7 g of a 15% by weight aqueous sodium persulfate solution and 1.5 g of a 2.5% by weight aqueous hydrogen peroxide solution. The kneader was stirred at maximum speed (98 rpm for the faster shaft, about 49 rpm for the slower shaft, ratio about 2:1). Immediately following the addition of hydrogen peroxide, the kneader jacket was heated with hot heat transfer medium at 80° C. 152 g of fine SAP dust having a degree of neutralization of 60 mol % and a particle size of less than 100 μm were added at an internal temperature of 60° C. On reaching the maximum temperature, the jacket heating was switched off and the kneader contents were allowed to react for a further 15 minutes in a supplementary reaction. The gel was cooled down to 65° C. and discharged. The gel was dried at 155° C. for 90 minutes using a loading of 700 g per tray in a circulating air drying cabinet. Following threefold grinding in a roll mill (Gebr. Baumeister LRC 125/70, gap widths 1000 μm, 600 μm, 400 μm), the polymer was sieved to obtain a size cut between 850 and 100 μm.

The Centrifuge Retention Capacity (CRC) of the water-absorbing polymers was 35.7 g/g.

1200 g of this polymer were transferred into a Gebr. Lödige laboratory mixer (M5R model). At room temperature, a mixture of 12 g of 1,2-propanediol, 1.2 g of diethylene glycol diglycidyl ether and 24 g of water was sprayed in via a first nozzle and 12 g of an aluminum sulfate solution (26.8% by weight of Al₂(SO₄)₃ in water) via a second nozzle. The mixer was then rapidly heated to 160° C. and maintained at 160° C. for 40 minutes. After cooling, the polymer was sieved to obtain a size cut between 850 and 100 μm.

The Centrifuge Retention Capacity (CRC) of the surface-postcrosslinked water-absorbing polymers was 29.2 g/g.

### Example 3

Production of Water-Absorbing Polymer (Degree of Neutralization 72 mol %)

4809 g of a 37.3% by weight aqueous sodium acrylate solution were mixed with 534 g of acrylic acid and 573 g of water and inertized with nitrogen. This mixture was filled into a nitrogen-inertized Werner & Pfleiderer LUK 8.0 K2 kneader (2 sigma shafts) and admixed in succession with 4.8 g of polyethylene glycol diacrylate 400 (diacrylate of a polyethylene glycol having an average molecular weight of 400 g/mol), 4.8 g of 15-tuply ethoxylated trimethylolpropane triacrylate, 4.4 g of a 1.0% by weight aqueous ascorbic acid solution, 18.1 g of a 15% by weight aqueous sodium persulfate solution and 3.9 g of a 3% by weight aqueous hydrogen peroxide solution. The kneader was stirred at maximum speed (98 rpm for the faster shaft, about 49 rpm for the slower shaft, ratio about 2:1). Immediately following the addition of hydrogen peroxide, the kneader jacket was heated with hot heat transfer medium at 80° C. On reaching the maximum temperature, the jacket heating was switched off and the kneader contents were allowed to react for a further 15 minutes in a supplementary reaction. The gel was cooled down to 65° C. and discharged. The gel was dried at 175° C. for 75 minutes using a loading of 700 g per tray in a circulating air drying cabinet. Following threefold grinding in a roll mill (Gebr. Baumeister LRC 125/70, gap widths 1000 μm, 600 μm, 400 μm), the polymer was sieved to obtain a size cut between 850 and 100 μm.

The Centrifuge Retention Capacity (CRC) of the water-absorbing polymers was 36.1 g/g.

1200 g of this polymer were transferred into a Gebr. Lödige laboratory mixer (M5R model). At room temperature, a mixture of 12 g of 1,2-propanediol, 1.3 g of diethylene glycol diglycidyl ether and 28 g of water was sprayed in via a first nozzle and 12 g of an aluminum sulfate solution (26.8% by weight of Al₂(SO₄)₃ in water) via a second nozzle. The mixer was then rapidly heated to 168° C. and maintained at 168° C. for 40 minutes. After cooling, the polymer was sieved to obtain a size cut between 850 and 100 μm.

The Centrifuge Retention Capacity (CRC) of the surface-postcrosslinked water-absorbing polymers was 30.0 g/g.

### Example 4

Portions of 100 g each of water-absorbing polymer from Example 1 were mixed in a tumble mixer with different amounts of sodium fluoride for 20 minutes at a time. Then the Centrifuge Retention Capacity (CRC) was measured.

The following table shows the measured results:

TABLE 1
sodium fluoride as urease inhibitor

| Concentration in composition | CRC |
|---|---|
| none | 28.5 g/g |
| 0.0500% by weight | 29.1 g/g |
| 0.0900% by weight | 28.5 g/g |
| 0.2000% by weight | 24.1 g/g |

### Example 5

Example 4 was repeated using hydroquinone instead of sodium fluoride.

TABLE 2
hydroquinone as urease inhibitor

| Concentration in composition | CRC |
|---|---|
| none | 28.5 g/g |
| 0.0050% by weight | 28.5 g/g |
| 0.0500% by weight | 28.7 g/g |
| 0.2000% by weight | 25.3 g/g |
| 1.0000% by weight | 23.4 g/g |

### Example 6

Example 4 was repeated using an 80% mixture of N-(n-butyl)thiophosphoramide and N-(n-propyl)thiophosphoramide (weight ratio 3:1) instead of sodium fluoride.

TABLE 3
N-(n-butyl)thiophosphoramide/N-(n-propyl)thiophosphoramide
as urease inhibitor

| Concentration in composition | CRC |
|---|---|
| none | 28.5 g/g |
| 0.0500% by weight | 29.0 g/g |
| 0.0900% by weight | 28.8 g/g |
| 0.2000% by weight | 25.9 g/g |

### Example 7

Portions of 100 g each of water-absorbing polymer from Examples 1 to 3 were mixed in a tumble mixer with different amounts of hydroquinone for 20 minutes at a time. Then the odor-inhibiting effect was measured.

The following table shows the measured results:

TABLE 4
hydroquinone as urease inhibitor (NH₃ after 6 hours)

| | Degree of neutralization | | |
|---|---|---|---|
| Concentration in composition | 55 mol % | 60 mol % | 72 mol % |
| none | 120 ppm | 208 ppm | 850 ppm |
| 0.0025% by weight | 91 ppm | 182 ppm | 800 ppm |
| 0.0050% by weight | 58 ppm | 78 ppm | 381 ppm |
| 0.0100% by weight | 29 ppm | 45 ppm | 214 ppm |
| 0.0200% by weight | 0 ppm | 10 ppm | 63 ppm |
| 0.0500% by weight | 0 ppm | 0 ppm | 15 ppm |
| 0.2000% by weight | 0 ppm | 0 ppm | 0 ppm |
| 1.0000% by weight | 0 ppm | 0 ppm | 0 ppm |

### Example 8

Example 7 was repeated using an 80% mixture of N-(n-butyl)thiophosphoramide and N-(n-propyl)thiophosphoramide (weight ratio 3:1) instead of hydroquinone.

TABLE 5
N-(n-Butyl)thiophosphoramide/N-(n-propyl)thiophosphoramide
as urease inhibitor (NH₃ after 6 hours)

Degree of neutralization

| Concentration in composition | 55 mol % | 72 mol % |
|---|---|---|
| none | 120 ppm | 850 ppm |
| 0.0100% by weight | 25 ppm | 49 ppm |
| 0.0300% by weight | 7 ppm | 31 ppm |
| 0.0500% by weight | 0 ppm | 6 ppm |
| 0.0900% by weight | 0 ppm | 0 ppm |
| 0.2000% by weight | 0 ppm | 0 ppm |

1. A composition comprising at least one water-absorbing polymer and at least one urease inhibitor, the polymer being a polymer bearing acid groups, the acid groups being from 50 to 65 mol % neutralized and the urease inhibitor content being in the range from 0.0001% to 0.1% by weight, based on the composition.

2. The composition according to claim 1 wherein the acid groups of the water-absorbing polymer are from 54 to 61 mol % neutralized and/or the urease inhibitor content is in the range from 0.015% to 0.045% by weight, based on the composition.

3. The composition according to claim 1 comprising at least 90% by weight of water-absorbing polymer.

4. The composition according to claim 1 wherein the water-absorbing polymer is a polymer based on a crosslinked acrylic acid.

5. The composition according to claim 1 wherein the water-absorbing polymer is in the form of surface-postcrosslinked particles.

6. A process for producing the composition defined in claim 1, which comprises at least one of the following steps:
i) mixing at least one urease inhibitor with at least one water-absorbing polymer,
ii) grinding at least one urease inhibitor together with at least one water-absorbing polymer,
iii) spraying at least one urease inhibitor onto at least one water-absorbing polymer,
iv) preparing the at least one water-absorbing polymer by solution polymerization of a monomer solution and dissolving or suspending at least one urease inhibitor in the monomer solution.

7. A process for producing the composition defined in claim 1, which comprises at least one of the following first stage (I) steps:
i) mixing at least one urease inhibitor with at least one water-absorbing polymer,
ii) grinding at least one urease inhibitor together with at least one water-absorbing polymer,
iii) spraying at least one urease inhibitor onto at least one water-absorbing polymer,
iv) preparing the at least one water-absorbing polymer by solution polymerization of a monomer solution and dissolving or suspending at least one urease inhibitor in the monomer solution,
and in a second stage (II) mixing the composition obtained in the first stage (I) according to i), ii), iii) and/or iv) together with at least one water-absorbing polymer.

8. The process according to claim 7 wherein the composition after stage (I) and before stage (II) comprises from 1% to 50% by weight of the at least one urease inhibitor, based on the composition.

9. A hygiene article comprising at least one composition according to claim 1.

10. The hygiene article according to claim 9 being a diaper or a pad for heavy or light incontinence, a sanitary napkin, a baby diaper, or a cat litter.
